(19) United States
(12) Patent Application Publication
(10) Pub. No.: US 2021/0236365 A1
(43) Pub. Date: Aug. 5, 2021

(54) THERAPY SYSTEM AND METHOD OF USING THE SAME

(71) Applicant: OXYZEN SYSTEMS LTD., RAMAT GAN (IL)

(72) Inventors: YEHUDA IDEL ZILBERMAN, ROSH HA'AYIN (IL); RONI SAPIR, TEL-AVIV (IL)

(21) Appl. No.: 17/272,313

(22) PCT Filed: Aug. 18, 2019
(86) PCT No.: PCT/IL2019/050921

§ 371 Date: Feb. 28, 2021

Related U.S. Application Data
(60) Provisional application No. 62/724,912, filed on Aug. 30, 2018.

(51) Int. Cl. A61G 10/00; A61N 5/06; A61H 1/00; A61F 7/00; G16H 20/30; G16H 40/67

(52) U.S. Cl. CPC A61G 10/00; A61N 5/06; A61H 1/005; A61N 2005/067; G16H 20/30; G16H 40/67; A61F 7/00

(57) ABSTRACT

The subject matter discloses a therapy system, comprising a body, a treatment module located inside the body, said treatment module comprising a plurality of treatment units, a management system coupled to the treatment module, comprising an interface module configured to receive information by users of the therapy system, a processing module for determining a treatment session to be performed by the treatment module based on the information received by the interface module, a local communication module for sending a command to the treatment module, said command defines the treatment session determined by the processing module. The body may include a treatment bed.

### FIELD OF THE INVENTION

The present invention relates to a therapy system, in which a user can get a treatment in an autonomous manner.

### BACKGROUND OF THE INVENTION

People are exposed to various mental and physical challenges that affect our lives, both in the physical aspect and mental aspects. Some solutions to these challenges are provided from the traditional medicine, such as medical drugs, medical diagnostics and the like. Other solutions may be by repeatedly meditating, visiting the masseuse, keeping work-life balance and the like.

Some of the solutions to handle the mental and physical challenges come from alternative medicine, which is defined as any practice that aims to achieve the healing effects of medicine, but which lacks biological plausibility and is untested or untestable is used together with functional medical treatment. The alternative sector is a highly profitable industry with a strong lobby, and faces far less regulation over the use and marketing. Its marketing often advertises the treatments as being “natural” or “holistic”, compared to those offered by “big pharma”.

People may use alternative treatments such as Acupuncture, Aromatherapy, Ayurvedic, medicine, Bowen technique, Homeopathy, Hypnotherapy, Massage when visiting a professional in alternative treatments. However, these treatments are often quite expensive as they require the professional's attention during the entire treatment. Due to the costs of personal alternative treatment, many people do not treat their body and mind in the amount and type they desire.

### SUMMARY OF THE INVENTION

It is an object of the subject matter to disclose a therapy system, comprising a body, a treatment module located inside the body, said treatment module comprising a plurality of treatment units, a management system coupled to the treatment module, comprising an interface module configured to receive information by users of the therapy system, a processing module for determining a treatment session to be performed by the treatment module based on the information received by the interface module, a local communication module for sending a command to the treatment module, said command defines the treatment session determined by the processing module.

In some cases, the body comprises a bottom section and a top section connected thereto, wherein the bottom section is affixed and the top section is movable relative to the bottom section, wherein the plurality of treatment units are placed in at least one of the bottom section and a top section.

In some cases, the top section comprises at least one treatment unit of the plurality of treatment units and the bottom section comprises at least one treatment unit of the plurality of treatment units. In some cases, the therapy system further comprises a connector for connecting the bottom section and the top section. In some cases, the bottom section comprises a treatment unit for vibrating the bottom section. In some cases, the bottom section comprises a treatment unit for heating the bottom section. In some cases, the top section comprises a treatment unit for emitting low-power laser signals. In some cases, the top section comprises a treatment unit for emitting illumination in a visible spectrum.

In some cases, the therapy system further comprises a sensor module coupled to the management system, wherein the sensor module is configured to collect environmental signals in the therapy system and send the environmental signals to the management system. In some cases, the interface module comprises a monitor and a graphic user interface, wherein the graphic user interface displays a plurality of optional treatment sessions on the monitor, wherein the interface module receives a selected treatment session by the user. In some cases, the therapy system further comprises a remote communication module configured to send signals to a destination located outside the therapy system. In some cases, the therapy system further comprises a payment terminal for receiving payment from the user.

In some cases, the management system further comprises a memory module for storing multiple treatment protocols, wherein the interface module is configured to display a menu of the multiple treatment protocols and receive the a selection of a single treatment protocol of the multiple treatment protocols from the user.

It is an object of the subject matter to disclose a computerized method for applying therapy to a user of a therapy system, said method comprising receiving information by the user of the therapy system, determining a treatment session to be performed on the user based on the received information, sending a command to a treatment module of the therapy system, said command defines the determined treatment session, applying the treatment session on the user by the treatment module of the therapy system.

In some cases, the method further comprises identifying the user and automatically determining the treatment session based on the user's identity. In some cases, the method further comprises collecting environmental measurements during the treatment session and updating an operation module of the plurality of treatment units of the treatment module based on the collected environmental measurements. In some cases, the information comprises a treatment protocol as defined by the user. In some cases, the information comprises a selection of a treatment protocol from a plurality of treatment protocol displayed on a monitor of the therapy system.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a management system for managing operations in a therapy system, according to exemplary embodiments of the present invention;

FIG. 2 is a block diagram showing the therapy module and its components, according to exemplary embodiments of the present invention;

FIG. 3 shows a method of managing a treatment session, according to exemplary embodiments of the present invention; and,

FIGS. 4A and 4B show a side view of the therapy system in an open state and a closed state, according to exemplary embodiments of the disclosed subject matter

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF THE INVENTION

The subject matter discloses a therapy system for applying treatments to a user. The therapy system comprises a surface on which the user lays, sits or stands, and a plurality of treatment units configured to provide the treatment to the user. The treatment units may provide functional treatments, such as supplying oxygen and/or radiation to the user. The treatment units may provide alternative treatments, such as aromatherapy, outputting sounds and the like. The therapy system comprises a computerized management system electrically coupled to the treatment units, the management system is configured to manage the treatments provided by the treatment units. The therapy system may be operated autonomously, without intervention of an operator. For example, when placed in a public place such as a mall, users may enter the therapy system, select a treatment session, lay on the treatment bed and enjoy the therapy.

The therapy system may be implemented as a closed compartment in a room, such that the user enters into the compartment in order to receive a therapy. The compartment may be located inside a residence, office, mall and the like. The therapy system may be implemented as a therapy bed, in which the user lays on the bed and receives the treatment.

The therapy system may be movable, for example when implemented as a bed that can be carried from one location to another. The therapy system may be connected to wheels to facilitate transport of the therapy system. The therapy system comprises disposable items, such as sheets on which the user lays, as well as hardware used to apply the treatment. Such hardware may include illumination modules, UV radiation emitters, audio speakers, a tube for supplying oxygen to the user, and the like.

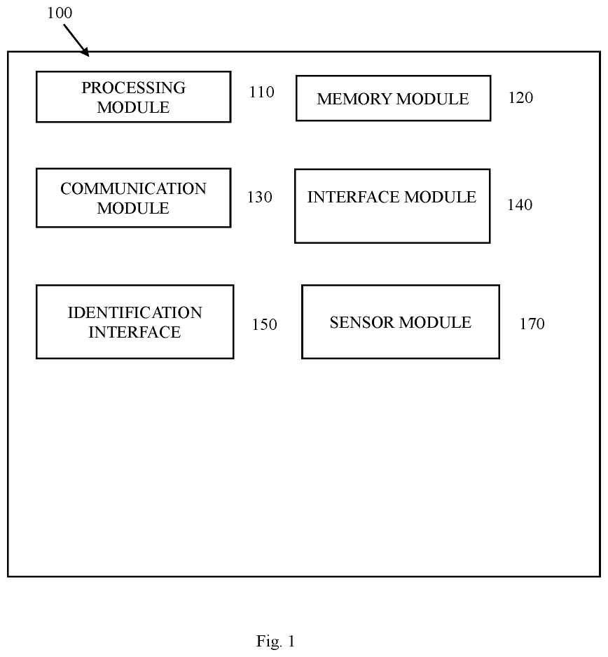

FIG. 1 shows a management system for managing operations in a therapy system, according to exemplary embodiments of the present invention. The management system 100 is electrically coupled to the treatment modules that apply the treatment to the user of the therapy system. The management system may be assembled inside a housing as a separate entity located near the body of the therapy system, where the user receives the treatment. In some other cases, the management system may be integrated with the body of the therapy system. The housing may have a window enabling users to directly view a monitor used to display information.

The management system 100 comprises a processing module 110 configured to determine properties associated with the functionality of the therapy system. The processing module 110 may determine properties of the treatment protocol for the next treatment session. The processing module 110 may be configured to determine properties based on the collected signals from sensors of the therapy system, or based on information received via interface module 140. The processing module 110 may determine the user's payment.

The management system 100 also comprises memory module 120 electrically coupled to the processing module 110. The memory module 120 is configured to store information related to the operation of the therapy system, such as user's personal information, therapy log of users of the therapy system, identification details of users of the therapy system and a set of rules used by the processing module 110 to manage the therapy system. The memory module 120 may store an adjustment set of rules configured to dictate how to change the operation of one or more treatment units of the therapy system during treatment session based on information collected by the sensor module 170. The set of rules may be stored in executable instructions accessed by the processing module 110 when receiving a request to calculate the treatment protocol for next treatment session.

The memory module 120 comprises one or more treatment protocols, each of the treatment protocols define the operation of the plurality of treatment units. The plurality of treatment units may operate concurrently, or one treatment unit operates at a time. At least one of the one or more treatment protocols comprises operation of two or more treatment units, in a manner that provides synergy between the operations of two or more treatment units. For example, treatment protocol #1 lasts 35 minutes, and dictates operation of the oxygen module for the entire treatment, as well as vibration at a specific frequency for between 18:30 and 22 minutes from the beginning of the treatment. In another example, treatment protocol #5 lasts 30 minutes, and dictates operation of the Infrared module for the first 3 minutes in a first radiation range, then the vibration module is used in combination with the Infrared module in a second radiation range for 7.5 minutes, and then oxygen and audio for the rest of the treatment. In some exemplary cases, additional protocols can be added to the memory module via the communication module 130, for example via a dedicated software operating on the management system 100. The treatment protocols may be assigned to identifiers or names, such that when displayed on a monitor, the user of the therapy system may select the treatment protocol that fits his/her desire.

The management system 100 also comprises a communication module 130, electrically coupled to the processing module 110. The communication module 130 is configured to exchange signals with communication units associated with the components in the therapy system. Such components may be the interface module 140, the treatment module, and the like.

The management system 100 also comprises interface module 140 configured to enable users to input information into the management system 100. The interface module 140 may be implemented as a touch-operated screen, keyboard, mouse and a combination thereof. The interface module 140 may comprise a monitor configured to display information to the user of the therapy system. The user may input his/her selection from a menu displayed on the monitor. For example, the processing module 110 determines that for a specific user, a specific menu is displayed on the monitor. The specific user then inputs his/her selection into the interface module 140 from the specific menu. The specific menu may include three (3) treatment options from a total of seven (7) treatment protocols operable by the therapy system.

The management system 100 also comprises an identification module 150 configured to enable users to identify at the therapy system. The identification module 150 may communicate with an identification terminal located at the therapy system. When the user arrives at the therapy system, he/she identifies at the identification terminal, for example by inputting information into the identification terminal of the identification interface 155, such as username and/or password, identification card, biometrical identification (i.e., voice recognition, face recognition, fingerprint recognition and more and more) or any another identification method. In some cases, identification may be assisted by an operator of the therapy system who collects the user's information and inputs it into the identification terminal.

When identification terminal collects the user's information, a communication unit of the identification terminal sends the user's information to the management system 100. The identification module 150 may comprise an identification communication module configured to exchange signals with the communication module 130.

The management system 100 may also comprise a sensor module 170 configured to collect environmental measurements inside and in the vicinity of the therapy system. The environmental measurements may include temperature, humidity, sound, smoke, materials, images and the like. The sensor module 170 may be coupled to the processing module 110, such that in case the environmental measurements collected by the sensor module 170 match a predefined rule or criteria, the processing module 110 will act upon said predefined rule. Actions of the processing module 110 may include adjusting the operation of one or more treatment units of the treatment modules, outputting an alert signal on the monitor of the interface module 140, emitting an audio signal indicating via a speaker and the like.

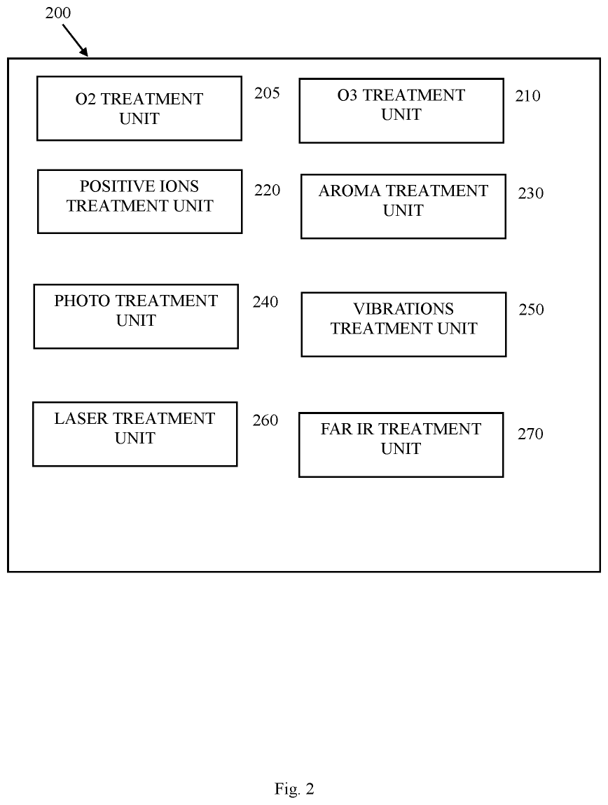

FIG. 2 is a block diagram showing the treatment module and its components, according to exemplary embodiments of the present invention. The treatment units in the treatment modules may be connected to a power grid, may have a power source such as a battery, or both.

The treatment module 200 comprises an oxygen treatment unit 205 configured to emit oxygen. The O₂ treatment unit 205 provides the user of the therapy system with oxygen therapy, also known as supplemental oxygen. The rate of oxygen provided by the oxygen treatment unit 205 may be standard, for example in the range of 3-10 liters per minute. In some cases, the oxygen treatment unit 205 may provide different rates for different treatment protocols.

The treatment module 200 may also comprise an ozone treatment unit 210 configured to emit Ozone gas. The ozone treatment unit 210 may emit the Ozone gas in a concentration range of 50-100 milligram per minute, and an emission rate in the range of 1-2 liters per minute.

The treatment module 200 may also comprise a negative ions treatment unit 220 for emitting negative ions into the body of the therapy system. For example, the outlet of the negative ions treatment unit 220 may be located between the top section and the bottom section of the therapy system's body.

The negative ions treatment unit 220 may dispense 0.1-20 million negative ions per second. The negative ions treatment unit 220 may be located in the central third of the therapy system's body, relatively proximal to the user's back and abdominal.

The treatment module 200 may also comprise an aroma therapy unit 230, configured to dispense fragrances inside the body of the therapy system. The aroma therapy unit 230 may comprise one or more tanks for storing gas or liquid dispensed via a tube. The management system sends a command to the aroma therapy unit 230, said command indicates which of the fragrances to be emitted by the aroma therapy unit 230. The tanks may store aromatic materials, such as essential oils, and other aroma compounds.

The treatment module 200 may also comprise a photo therapy unit 240 for emitting light inside the body of the therapy system. The light's properties, such as wavelength, frequency, amplitude and additional properties, may be included in the treatment protocols stored in the memory module 120 of the management system 100. The photo therapy unit 240 comprises one or more illumination modules, such as light emitting diodes (LEDs), fluorescent lamps, dichroic lamps, laser emitters or other illuminators desired by a person skilled in the art.

The treatment module 200 may also comprise a vibration treatment unit 250 for vibrating a surface on which the user lays or sits. The vibration treatment unit 250 may be placed inside or under the bottom side of the therapy system. The vibration treatment unit 250 may produce vertical vibrations, horizontal vibrations and a combination thereof.

The treatment module 200 may also comprise a laser treatment unit 260 for emitting low-power lasers towards the user. The low power laser may have electrical power in the range of 1-5 mW. The laser wavelength may be in the range of 400-700 nm.

The treatment module 200 may also comprise a far infra-red (IR) therapy unit 270 for emitting IR radiation towards the user of the therapy system. The properties of the radiation, such as the amplitude and wavelength, may be defined for a treatment protocol stored in the memory module 120.

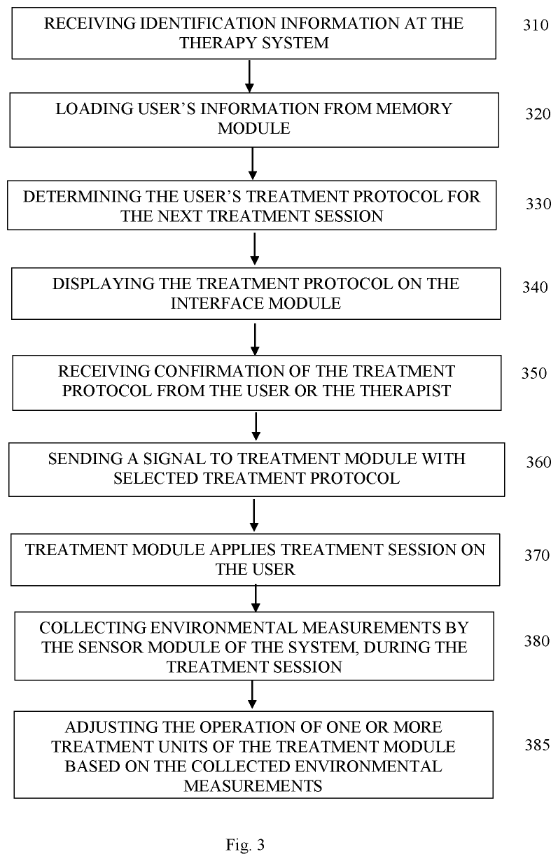

FIG. 3 shows a method of managing a treatment session, according to exemplary embodiments of the present invention. The treatment session is performed at a certain time and place based on a treatment protocol as disclosed above. The treatment protocol defines the operation plan of the treatment units. The treatment session is defined as a specific session. For example, two different treatment sessions are provided by the therapy system for a single user who uses the therapy system twice, for example in two subsequent days. The treatment protocol may be selected with respect to the user's identification, user's requirements and the range of treatment protocols available on the therapy system.

Step 310 discloses receiving identification information from the user of the therapy system. In some cases, the user's identification information may include credentials collected by an identification terminal or by a person operating the therapy system. In such cases, the user may input the identification information into an identification terminal. For example, identification information may be a username, identification card, biometrical identification (i.e., voice recognition, face recognition, fingerprint recognition and the like) or any other identification method, mechanism or technique desired by a person having ordinary skills in the art.

Step 320 discloses loading user's information from the memory module of the therapy system. The memory module may be an integral part of the system, or be part of a remote device, such as an online system. The user's information may be personal information such as the user's name, age, gender, weight, medical background and the like. The user's information may also include a set of business rules that match the user, such as the user's treatment history in the therapy system, whether or not the user has an annual membership at the therapy system, and the like.

Step 330 discloses determining the user's treatment protocol for the next treatment session. The next treatment session may be determined automatically by the management system of the therapy system based on user's information loaded from the memory unit as well as additional rules and information. In some other cases, the user's treatment protocol for the next treatment session may be determined by an operator of the therapy system and/or a therapist who assists the user of the therapy system. In some cases, the user of the therapy system may use the interface module to create a treatment protocol based on the treatment units of the therapy system. In such cases, the processing module may define limitations for the treatment protocol in case operation of one treatment unit contradicts with the operation of another treatment unit.

Step 340 discloses the displaying the treatment protocol in the interface module. The processing module may send the treatment protocol determined in the step 330 to the interface module. The determined treatment protocol is presented on the interface module.

Step 350 discloses receiving the user or the therapist confirmation on the treatment protocol. In some cases, the determined treatment protocol is applied only after confirmation by the user or the therapist. In some cases, the user and/or therapist may change the treatment protocol using the interface module, such as a keyboard and touch-operated screen.

Step 360 discloses sending a command to treatment module with next treatment session determined above. The command may include the ordering sequence of therapy units that will be used in the next treatment session and a time duration of using each therapy unit. The commands may be sent via Wi-Fi, Bluetooth, via wired channels and the like.

Step 370 discloses applying treatment session on user by therapy module. The treatment session is based on the determined treatment protocol. The treatment session is performed by at least one treatment unit of the treatment module.

Step 380 discloses collecting environmental measurements by the sensor module of the system, during the treatment session. In such cases, the environmental measurements comprise the user's biometric information. For example, the biometric information may be pulse, skin color, skin temperature and the like. Step 385 discloses adjusting the operation of one or more treatment units of the treatment module based on the collected environmental measurements.

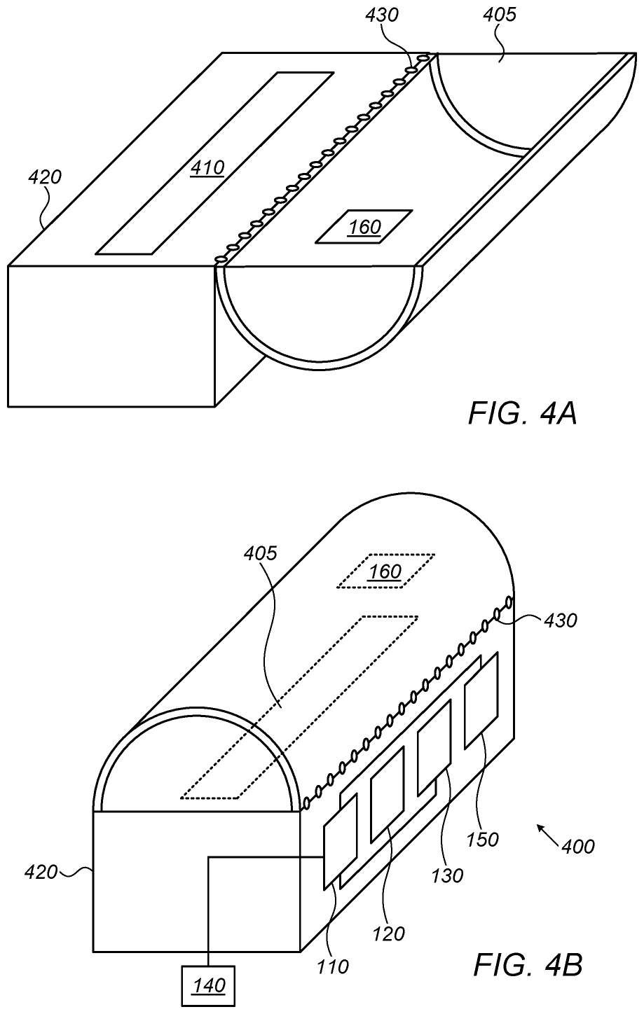

FIGS. 4A and 4B show a side view of the therapy system in an open state and a closed state, according to exemplary embodiments of the disclosed subject matter. The therapy system comprises a body, having a top section 405 and a bottom section 420. The bottom section 420 may be placed directly on the floor/ground, or may be coupled to legs mounted on the floor. The height of the legs may be in the range of 70-120 centimeters. The bottom section 420 has an upper surface 410 on which the user lays during the treatment session. The treatment units 160 may be placed in at least one of the top section 405 and the bottom section 420. For example, the vibration treatment unit may be integrated inside the bottom section 420, as the user lays on top of the bottom section 420.

In some exemplary cases, the top section 405 is movable relative to the bottom section 420. The top section 405 and the bottom section 420 may be connected to each other via a connector, such as hinges 430 that enable circular movement of the top section 405 relative to the bottom section 420. In some other cases, the top section 405 is removable from the bottom section 420 and can be mounted on top of the bottom section 420 in a manner that enables the user to be placed inside the body of the therapy system.

The closed position is defined as a case in which the top section 405 and the bottom section 420 cover most of the user's body, while the open position is defined by a minimal distance between the top section 405 and the bottom section 420. In some exemplary cases, the top section 405 has a circular cross section, leaving room to the user to comfortably lay on the upper surface 410 of the bottom section 420.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A therapy system, comprising:
a body;
a treatment module located inside the body, said treatment module comprising a plurality of treatment units,
a management system coupled to the treatment module, comprising:
an interface module configured to receive information by users of the therapy system;
a processing module for determining a treatment session to be performed by the treatment module based on the information received by the interface module;
a local communication module for sending a command to the treatment module, said command defines the treatment session determined by the processing module.

2. The therapy system of claim 1, wherein the body comprises a bottom section and a top section connected thereto, wherein the bottom section is affixed and the top section is movable relative to the bottom section, wherein the plurality of treatment units are placed in at least one of the bottom section and a top section.

3. The therapy system of claim 2, wherein the top section comprises at least one treatment unit of the plurality of treatment units and the bottom section comprises at least one treatment unit of the plurality of treatment units.

4. The therapy system of claim 2, further comprises a connector for connecting the bottom section and the top section.

5. The therapy system of claim 2, wherein the bottom section comprises a treatment unit for vibrating the bottom section.

6. The therapy system of claim 2, wherein the bottom section comprises a treatment unit for heating the bottom section.

7. The therapy system of claim 2, wherein the top section comprises a treatment unit for emitting low-power laser signals.

8. The therapy system of claim 2, wherein the top section comprises a treatment unit for emitting illumination in a visible spectrum.

9. The therapy system of claim 1, further comprises a sensor module coupled to the management system, wherein the sensor module is configured to collect environmental signals in the therapy system and send the environmental signals to the management system.

10. The therapy system of claim 1, wherein the interface module comprises a monitor and a graphic user interface, wherein the graphic user interface displays a plurality of optional treatment sessions on the monitor, wherein the interface module receives a selected treatment session by the user.

11. The therapy system of claim 1, further comprises a remote communication module configured to send signals to a destination located outside the therapy system.

12. The therapy system of claim 1, further comprises a payment terminal for receiving payment from the user.

13. The therapy system of claim 1, wherein the management system further comprises a memory module for storing multiple treatment protocols, wherein the interface module is configured to display a menu of the multiple treatment protocols and receive the a selection of a single treatment protocol of the multiple treatment protocols from the user.

14. A computerized method for applying therapy to a user of a therapy system, said method comprising:
receiving information by the user of the therapy system;
determining a treatment session to be performed on the user based on the received information;
sending a command to a treatment module of the therapy system, said command defines the determined treatment session;
applying the treatment session on the user by the treatment module of the therapy system.

15. The method of claim 14, further comprises identifying the user and automatically determining the treatment session based on the user's identity.

16. The method of claim 14, further comprises collecting environmental measurements during the treatment session and updating an operation module of the plurality of treatment units of the treatment module based on the collected environmental measurements.

17. The method of claim 14, wherein the information comprises a treatment protocol as defined by the user.

18. The method of claim 14, wherein the information comprises a selection of a treatment protocol from a plurality of treatment protocol displayed on a monitor of the therapy system.
